(19) United States
(12) Patent Application Publication
(10) Pub. No.: US 2016/0030003 A1
(43) Pub. Date: Feb. 4, 2016

(54) ULTRASONIC DIAGNOSTIC DEVICE AND METHOD FOR SUPPORTING SYNCHRONOUS SCANNING WITH MULTIPLE PROBES

(71) Applicant: SHENZHEN MINDRAY BIO-MEDICAL ELECTRONICS CO., LTD., Shenzhen (CN)

(72) Inventors: Shuo Liu, Shenzhen (CN); Yong Li, Shenzhen (CN)

(73) Assignee: SHENZHEN MINDRAY BIO-MEDICAL ELECTRONICS CO., LTD.

(21) Appl. No.: 14/884,490

(22) Filed: Oct. 15, 2015

Related U.S. Application Data
(63) Continuation of application No. PCT/CN2013/083107, filed on Sep. 9, 2013.

(30) Foreign Application Priority Data
Apr. 16, 2013 (CN) 201310130584.X

(51) Int. Cl. A61B 8/00; A61B 8/14; A61B 8/08

(52) U.S. Cl. CPC A61B 8/4477; A61B 8/463; A61B 8/5207; A61B 8/42; A61B 8/54; A61B 8/4483; A61B 8/145

(57) ABSTRACT

Ultrasonic diagnostic devices and methods for supporting synchronous scanning are provided in this disclosure. The ultrasonic diagnostic device can include a display module, an imaging system and multiple probes. The multiple probes are capable of being attached to different positions on a body surface of a patient, such that synchronous and real-time scanning can be performed by the multiple probes for body parts corresponding to those different positions on the patient's body surface. Echo signals can be obtained by the multiple probes through scanning, and may then be transmitted to the imaging system. The imaging system can convert the multiple echo signals transmitted from the multiple probes into multiple ultrasonic images. The display module may be coupled to the imaging system. It can receive the multiple ultrasonic images processed and outputted by the imaging system, and display these images synchronously.

### CROSS-REFERENCE

This application is a continuation of Patent Cooperation Treaty Application No. PCT/CN2013/083107, filed Sep. 9, 2013, which is hereby incorporated by reference.

### TECHNICAL FIELD

This disclosure relates to the medical equipment field, and particularly to ultrasonic diagnostic devices and methods for supporting synchronous scanning with multiple probes.

### BACKGROUND

A probe is an important component of an ultrasonic diagnostic device, which converts electrical signals into sound signals to be emitted into a human body, and convert sound signals reflected from human tissue back into electrical signals to be transmitted to a signal processing unit of the ultrasonic diagnostic device for imaging. Ultrasonic diagnostic devices are widely used in the clinical field, and probes with different shapes and working frequencies have been applied in clinical applications.

For the purpose of matching with corresponding diagnostic parts, the probes may be designed to have different shapes and working frequencies according to depth, shape and structure of various diagnostic parts. During a diagnostic process, a doctor may be able to scan one single part of a patient at a certain moment when holding a probe by hand. Therefore, the doctor may often have to switch the probe so as to perform a complete ultrasonic diagnosis on the patient's different body parts. For example, a phased array probe may be first used for heart scanning, and a linear array probe may be then used for peripheral vessel scanning after switching probes.

Although the traditional ultrasonic diagnostic device can be connected with multiple probes through multiple slots (one-to-one connection), a single probe can be activated at a certain time instant, namely only one probe can be used for scanning imaging. For this reason, when the doctor needs to perform scanning imaging on different parts using different probes, the working probes may have to be switched in sequence so as to successively obtain image data of those different parts.

Up to now, synchronous scanning along different sections can only be realized by a biplane probe. The biplane probe may use two sound heads (referred to sound head A and sound head B in FIG. 1) for synchronous scanning along different sections. For a transrectal prostate examination, synchronous scanning can be simultaneously carried out along a vertical section and a cross section. Those two sound heads are arranged in one probe, and thus the different sections for the synchronous scanning are close to each other, which cannot meet wider clinical demands.

European patent 0528693A1 notes an ultrasonic diagnostic device supporting multiple probes to be connected to one slot. In this way, this ultrasonic diagnostic device can be simultaneously connected with the probes of which the number exceeds that of the slot. In the technical solutions disclosed in this patent, the ultrasonic diagnostic system consists of a host, a connector and several probes. The connector is a pair of plug and slot connected with the probes and the host, and a primary connector and a secondary connector are included in the probe structure. The primary connector is connected to the host or another probe; the secondary connector is connected with the primary connector and the connector of another probe through an interconnection cable, or the secondary connector can be connected with the probe branching from the interconnection cable (for connecting the primary and the secondary connectors).

In this patent, one slot can connect with multiple probes in the ultrasonic diagnostic device. When the number of probes is greater than that of a host slot, all the probes can still be connected with the host simultaneously, and thus there is no need to replace the probes by inserting them in and removing them from the slot during usage.

Those technologies in the prior patent are limited in their application range, and they have the following drawbacks:

The biplane probe has two sound heads for the synchronous scanning along different sections. However, since the two sound heads are arranged in one probe, the different sections for the synchronous scanning are too close to each other, which cannot meet wider clinical demands.

European patent 0528693A1 discloses an ultrasonic diagnostic device supporting multiple probes to be connected to one slot. Although this device can be simultaneously connected with the probes of which the number exceeds that of the slot, it may only enable the connection between the multiple probes and the system rather than supporting synchronous working and scanning of the multiple probes, which cannot meet the doctor's demands on concurrent diagnosis of different body parts.

### SUMMARY

Aiming at the above-described drawbacks in the prior art, ultrasonic diagnostic devices and methods for supporting synchronous scanning with multiple probes are provided in this disclosure. The devices and methods can support simultaneous and independent working of the multiple probes at the same time so that the ultrasonic diagnostic devices can simultaneously obtain respective scan image data of different probes, thereby meeting demands on concurrent diagnosis of different body parts.

In one aspect, an ultrasonic diagnostic device can include a display module, an imaging system and multiple probes.

The multiple probes are configured to be attached to different positions on a body surface of a patient, such that synchronous and real-time scanning can be performed by the multiple probes for different body parts corresponding to those different positions on the patient's body surface. Echo signals can be obtained by the multiple probes through scanning, and may then be transmitted to the imaging system.

The imaging system can convert the multiple echo signals transmitted from the multiple probes into multiple ultrasonic images.

The display module may be coupled to the imaging system. It can receive the multiple ultrasonic images processed and outputted by the imaging system, and display the processed ultrasonic images synchronously.

In some embodiments, each probe can be tightly attached to a respective fixed position on the body surface of the patient so that the scanning can be performed by each probe for the patient at the respective fixed position along a same section.

In some embodiments, the ultrasonic diagnostic device may further include one or more slots and one or more probe high-voltage switches, where the quantity of the one or more probe high-voltage switches may be equal to that of the one or more slots. The one or more slots can be used for insertion connection with the multiple probes. The one or more probe high-voltage switches can be used for controlling the multiple probes to be switched during repetition time intervals of scanning pulses, and thus alternating scanning can be performed by the multiple probes for the different body parts corresponding to those different positions on the body surface of the patient according to a preset scanning sequence.

In some embodiments, the preset scanning sequence can be defined as follows: the alternating scanning may be successively performed per scan line by the multiple probes for the different body parts of the patient.

In some embodiments, the preset scanning sequence can be defined as follows: the alternating scanning may be successively performed per frame by the multiple probes for the different body parts of the patient.

In some embodiments, each probe may include multiple array elements and one or more array element high-voltage switches corresponding to the multiple array elements. The array elements of each probe can be controlled by the one or more corresponding array element high-voltage switches arranged within the probe to perform the alternating scanning for the body part corresponding to the position on the body surface where each probe is attached.

In some embodiments, the probe high-voltage switch and the array element high-voltage switches can be controlled by a control circuit.

In some embodiments, the imaging system can perform digital processing on the multiple echo signals to obtain digital processing signals. The multiple ultrasonic images can be obtained based on the digital processing signals and a selected imaging mode. The imaging mode supported in the imaging system may be at least one of B imaging mode, M imaging mode, color imaging mode, pulse wave (PW) imaging mode, elasticity imaging mode, three-dimensional (3D) imaging mode and four-dimensional (4D) imaging mode.

In some embodiments, the ultrasonic diagnostic device can also include an operation panel for receiving a triggering signal.

In some embodiments, the display module can include multiple display windows. The multiple display windows can be used for displaying the multiple ultrasonic images in a real-time and synchronous way when the operation panel receives the triggering signal, where the multiple ultrasonic images may be obtained by the imaging system according to the multiple echo signals based on the selected imaging mode.

In some embodiment, the quantity of the multiple probes is greater than that of the one or more slots.

In another aspect, an ultrasonic diagnostic method realized by the afore-described ultrasonic diagnostic device can be provided, which may include the following steps:

performing synchronous and real-time scanning for different body parts corresponding to different positions on a body surface of a patient by multiple probes to obtain multiple echo signals, and transmitting the echo signals from the multiple probes to an imaging system;

converting the multiple echo signals transmitted from the multiple probes into multiple ultrasonic images by the imaging system; and

receiving the multiple ultrasonic images processed and outputted by the imaging system and displaying these images synchronously on a display module.

In some embodiments, the method can further include: controlling the multiple probes to be switched during repetition time intervals of scanning pulses by multiple probe high-voltage switches so that alternating scanning can be performed for the different body parts corresponding to those different positions on the body surface of the patient according to a preset scanning sequence.

In some embodiments, the preset scanning sequence can be defined as follows: the alternating scanning may be successively performed per scan line by the multiple probes for the different body parts of the patient.

In some embodiments, the preset scanning sequence can be defined as follows: the alternating scanning may be successively performed per frame by the multiple probes for the different body parts of the patient.

In some embodiments, the method may also include controlling multiple array elements of each probe by one or more array element high-voltage switches arranged within each probe to make the alternating scanning for the body part corresponding to the position on the body surface where each probe is attached.

In some embodiments, the probe high-voltage switch and the array element high-voltage switches can be controlled to be switched on or off by a control circuit.

In some embodiments, converting the multiple echo signals transmitted from the multiple probes into the multiple ultrasonic images by the imaging system can include:

performing digital processing on the multiple echo signals to obtain digital processing signals by the imaging system, and obtaining the multiple ultrasonic images based on the digital processing signals and a selected imaging mode. The imaging mode supported in the imaging system may be at least one of B imaging mode, M imaging mode, color imaging mode, PW imaging mode, elasticity imaging mode, 3D imaging mode and 4D imaging mode.

In the embodiments of this disclosure, multiple slots connected with multiple probes can be arranged on the ultrasonic diagnostic device. The probes can realize the synchronous and real-time scanning so as to perform the ultrasonic scanning and monitoring for a plurality of body parts of a test subject.

The probes used in the embodiments of this disclosure can be attached to a patient's body surface for a long time. This can ensure that each scanning for the respective probe is carried out along the same section so as to obtain more accurate ultrasonic images and avoid sound power risk caused by continuous scanning.

### BRIEF DESCRIPTION OF THE DRAWINGS

For illustrating embodiments of this disclosure or technical solutions in prior art more clearly, some figures for describing the embodiments or the prior art will be briefly described below. It is apparent that the figures in the following descriptions are only some examples of this disclosure. The ordinary skilled person in the art can obtain other figures according to these figures without paying any creative efforts.

FIG. 1 is a schematic diagram for a biplane probe in prior art;

FIG. 2 is a structure diagram for an ultrasonic diagnostic device according to an embodiment of this disclosure;

FIG. 3 is a schematic diagram illustrating synchronous displays of a display module of an ultrasonic diagnostic device in this disclosure;

FIG. 4 is a schematic diagram illustrating synchronous scanning of an ultrasonic diagnostic device in this disclosure;

FIG. 5 is a schematic diagram illustrating the working principle of high-voltage switches of an ultrasonic diagnostic device in this disclosure;

FIG. 6 is a schematic diagram illustrating scanning sequences during synchronous scanning of multiple probes of an ultrasonic diagnostic device in this disclosure;

FIG. 7 is a schematic diagram illustrating scanning sequences when multiple probes of an ultrasonic diagnostic device perform different imaging modes in this disclosure;

FIG. 8 is a flow chart for an ultrasonic diagnostic method according to a first embodiment of this disclosure;

FIG. 9 is a flow chart for an ultrasonic diagnostic method according to a second embodiment of this disclosure; and

FIG. 10 is a flow chart for an ultrasonic diagnostic method according to a third embodiment of this disclosure.

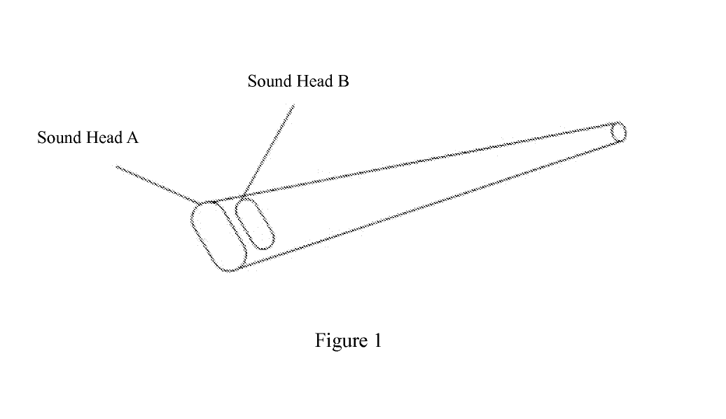

### DETAILED DESCRIPTION

Technical solutions in embodiments of this disclosure will be described clearly and completely below with reference to figures of the embodiments of this disclosure. Obviously, those embodiments described below are only a part rather than the whole of the embodiments of this disclosure. Based on the embodiments in this disclosure, all other embodiments obtained by the ordinary skilled person in the art without paying creative efforts can be included in the protection scope of this disclosure.

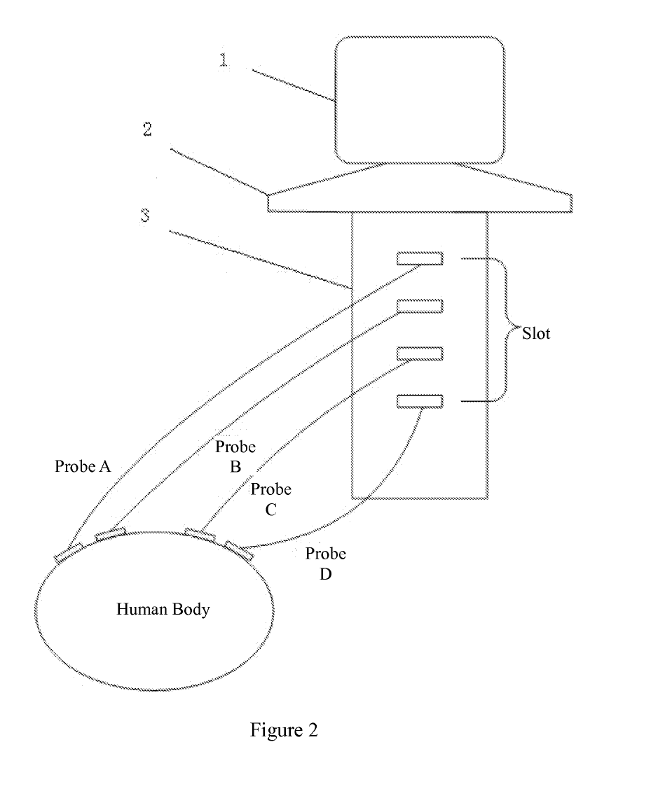

Ultrasonic diagnostic devices supporting synchronous scanning with multiple probes are provided in various embodiments of this disclosure, which will be described with reference to FIGS. 2-7 below.

Referring to FIG. 2, an ultrasonic diagnostic device supporting synchronous scanning with multiple probes provided in an embodiment of this disclosure may include a display module 1, an imaging system 3 and multiple probes (probe A, probe B, probe C and probe D as shown in the figure). This device can also include an operation panel 2 and slots. Although FIG. 2 includes multiple slots as an example, there can be one or more slots in other implementations. The multiple probes can be connected to the slots. The multiple probes may be configured to be attached to different positions on a body surface of a patient. In this way, synchronous and real-time scanning can be performed for different body parts corresponding to those different positions on the body surface of the patient through the multiple probes. Echo signals obtained through the scanning can be sent back to the imaging system 3 by the multiple probes. In a preferred implementation, the quantity of the multiple probes is larger than or equal to that of the slots. In an alternative embodiment, the slots can be connected with the multiple probes by an adapter when the slots are fewer than the probes (such as one single slot).

In some embodiments, each probe can be directly and tightly attached to a respectively fixed position on the body surface of the patient, so that the scanning can be performed by each probe for the patient at the respective fixed position along a same section. In this way, it can be ensured that each scanning for the respective probe is performed along the same section, thereby obtaining more accurate ultrasonic images, avoiding sound power risk caused by continuous scanning, and preventing discomfort of transesophageal probe in prior art.

The imaging system 3 can convert the multiple echo signals transmitted back from the multiple probes into multiple ultrasonic images.

The display module 1 may be coupled to the imaging system 3. It can receive the multiple ultrasonic images processed and outputted by the imaging system 3, and display these images synchronously.

It should be noted that the display module 1 in specific implementations can be a display device/module of a desktop or a portable or a hand-held ultrasonic device.

The operation panel 2 can be configured to receive a triggering signal. The display module 1 may include a plurality of display windows for displaying the multiple ultrasonic images in a real-time and synchronous way when the operation panel 2 receives the triggering signal, where the multiple ultrasonic images can be obtained by the imaging system 3 according to the multiple echo signals and the selected imaging mode.

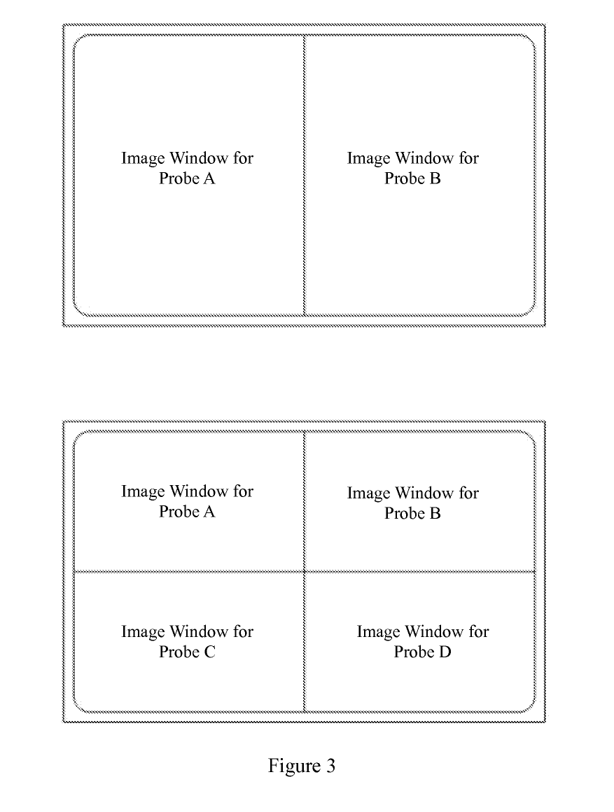

Referring to FIG. 3, when using two probes, namely probe A and probe B, for concurrent scanning, there may be two corresponding display windows: an image window for probe A and an image window for probe B. When using four probes, namely probe A, probe B, probe C and probe D, for concurrent scanning, there may be four corresponding display windows: an image window for the probe A, an image window for the probe B, an image window for the probe C and an image window for the probe D. When n probes are used for concurrent scanning, there will be n display windows correspondingly.

In this disclosure, the ultrasonic diagnostic device may also include one or more probe high-voltage switches so that the multiple probes of the ultrasonic diagnostic device provided in this disclosure can achieve the synchronous scanning.

The quantity of the one or more probe high-voltage switches is equal to that of the one or more slots. The probe high-voltage switch can be used for controlling the multiple probes to be switched during repetition time intervals of scanning pulses, so that alternate scanning can be performed for the different body parts corresponding to those different positions on the body surface of the patient according to a preset scanning sequence.

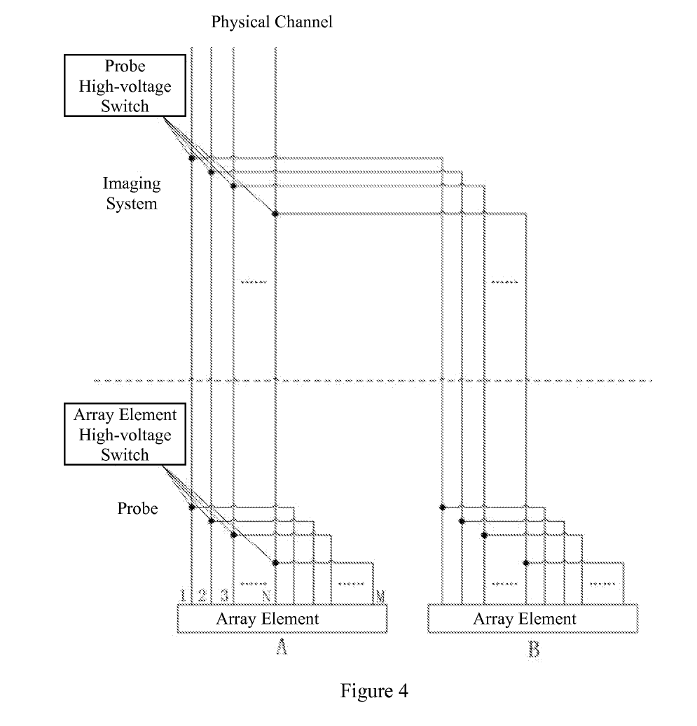

In addition, each probe may include a plurality of array elements. As shown in FIG. 4, for example, the probe A may include array elements 1, 2, 3 . . . N . . . M. Assuming the number of the array elements in each probe is greater than that of the slots (i.e., physical channel), each probe may further include one or more array element high-voltage switches. The array elements of each probe can be controlled by the one or more array element high-voltage switches arranged within each probe to perform the alternating scanning for the body part corresponding to the position where each probe is respectively attached.

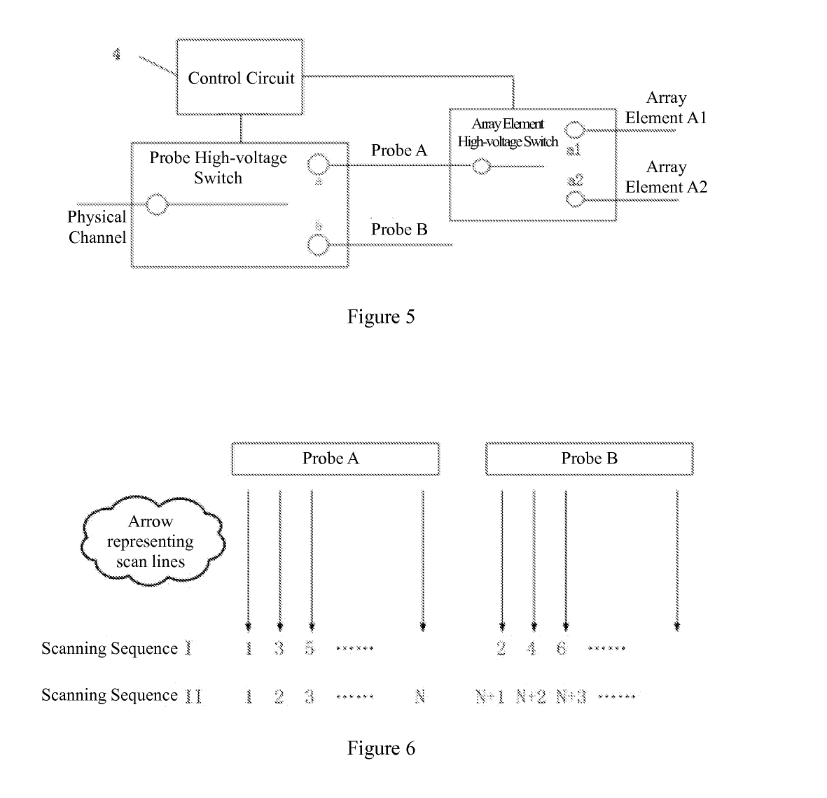

The one or more probe high-voltage switches and the one or more array element high-voltage switches can be controlled by a control circuit, such as the control circuit 4 shown in FIG. 5. Under the control of the control circuit 4, when the probe high-voltage switch is switched to a contact b of the probe B, the probe B can be connected with the physical channel, so that the probe B may start to work. Similarly, when the array element high-voltage switch is switched by the control circuit 4 to a contact a1 of an array element A1 of the probe A, the array element A1 of the probe A can be connected with the probe A, so that the array element A1 of the probe A may start to work; when the array element high-voltage switch is switched by the control circuit 4 to a contact a2 of an array element A2 of the probe A, the array element A2 of the probe A can be connected with the probe A, so that the array element A2 of the probe A may start to work.

It should be noted that a magnitude of the switching time may be a few microseconds for the probe high-voltage switch or the array element high-voltage switch. In this case, the probe switching can be completed during repetition time intervals of its normal scanning pulses. This is different from a conventional probe switching, which may need to use a relay and thus take too much time for switching.

In order to support the probe switching during the repetition time intervals of its normal scanning pulses, two scanning sequences described hereinafter are provided in this disclosure.

A first preset scanning sequence can be defined as follows: the alternating scanning may be successively performed per scan line by the multiple probes for the different body parts of the patient.

Specifically, a first probe of the multiple probes can first scan along a first scan line of the first probe through the body part corresponding to the position on the body surface to which the first probe is attached, and a second probe can then scan along a first scan line of the first probe through the body part corresponding to the position on the body surface to which the second probe is attached, and the scanning is carried out in a similar way until a last probe of the multiple probes can scan along a first scan line of the last probe through the body part corresponding to the position on the body surface to which the last probe is attached. After that, the first probe of the multiple probes can start to scan along a second scan line of the first probe through its corresponding body part, the second probe of the multiple probes can then scan along a second scan line of the second probe through its corresponding body part and so on. Such scanning sequence can be repeated until the multiple probes can respectively obtain a complete frame image for their corresponding body parts.

A second preset scanning sequence can be defined as follows: the alternating scanning may be successively performed per frame by the multiple probes for the different body parts of the patient.

Specifically, a first probe of the multiple probes can first scan its corresponding body part to obtain a frame image, and a second probe can then scan its corresponding body part to obtain another frame image, and the scanning sequence can be repeated until a last probe of the multiple probes scans its corresponding body part and obtains a frame image.

Hereinafter, two probes (probe A and probe B) may be taken as an example for illustration with reference to FIG. 6. The probe A can be attached to a position A on the body surface of the patient, while the probe B can be attached to a position B on the body surface of the patient.

Under the first scanning sequence, the probe A may scan a body part corresponding to the position A along a first scan line of the probe A, the probe B may then scan a body part corresponding to the position B along a first scan line of the probe B, the probe A may subsequently scan the body part corresponding to the position A along a second scan line of the probe A, and the probe B may scan the body part corresponding to the position B along a second scan line of the probe B and so on. The alternating scanning between the probes A and B can be repeated in sequence, until the probe A completes the scanning along all the scan lines and obtains a frame image of the body part corresponding to the position A by combining all these scan lines, and until the probe B completes the scanning along all the scan lines at the part B and obtains a frame image of the part B by combining all these scan lines. Such scanning sequence can be performed repeatedly, such that the probe A may obtain multiple frame images for the body part corresponding to the position A and the probe B may obtain multiple frame images for the body part corresponding to the position B.

Under the second scanning sequence, the probe A can first scan a body part corresponding to the position A to obtain a frame image following which the probe B may scan a body part corresponding to the position B to obtain another frame image; the probe A may then make a frame scanning once again while the probe B may subsequently make a frame scanning following the probe A and so on. Such scanning sequence can be repeated, such that the probe A may obtain multiple frame images for the body part corresponding to the position A and the probe B may obtain multiple frame images for the body part corresponding to the position B.

Besides, the probe high-voltage switch and the array element high-voltage switch in this disclosure can adjust the scanning sequence of the multiple probes, so that the multiple probes can support the synchronous scanning under different imaging modes. Each probe can flexibly select an imaging mode, where the imaging mode may be at least one of B (brightness) imaging mode, M (motion, sequence diagram for multipoint motion in single-dimensional space) imaging mode, color imaging mode, pulse wave (PW) imaging mode, elasticity imaging mode, three-dimensional (3D) imaging mode and four-dimensional (4D) imaging mode.

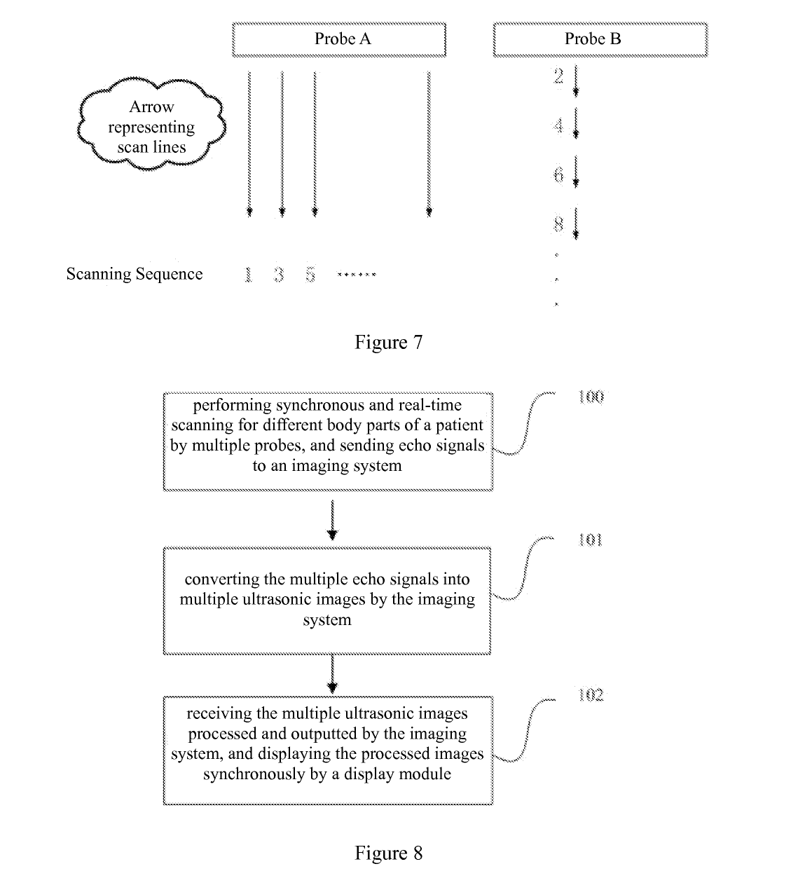

In an example, the probe A can select the B imaging mode while the probe B can select the M imaging mode. Their scanning sequences are shown in FIG. 7.

Correspondingly, the imaging system may convert the multiple echo signals transmitted back from the multiple probes into the multiple ultrasonic images through the following way: performing digital processing on the multiple echo signals to obtain digital processing signals, and obtaining the multiple ultrasonic images according to the digital processing signals and the selected imaging mode. The imaging mode supported in the imaging system may be at least one of B (brightness) imaging mode, M (motion, sequence diagram for multipoint motion in single-dimensional space) imaging mode, color imaging mode, PW imaging mode, elasticity imaging mode, 3D imaging mode and 4D imaging mode.

An ultrasonic diagnostic method is also provided in this disclosure, which can be implemented in the afore-described ultrasonic diagnostic device. FIG. 8 is a flow chart for an ultrasonic diagnostic method according to a first embodiment. This method may include the following steps (steps 100-102).

In step 100, multiple probes can be used for performing synchronous and real-time scanning for different body parts corresponding to different positions on a body surface of a patient to obtain multiple echo signals, and the multiple echo signals can then be transmitted from the multiple probes to an imaging system.

In step 101, the multiple echo signals transmitted from the multiple probes can be converted into multiple ultrasonic images by the imaging system.

In step 102, when the multiple ultrasonic images are processed and outputted by the imaging system, a display module can receive the processed multiple ultrasonic images and display them synchronously.

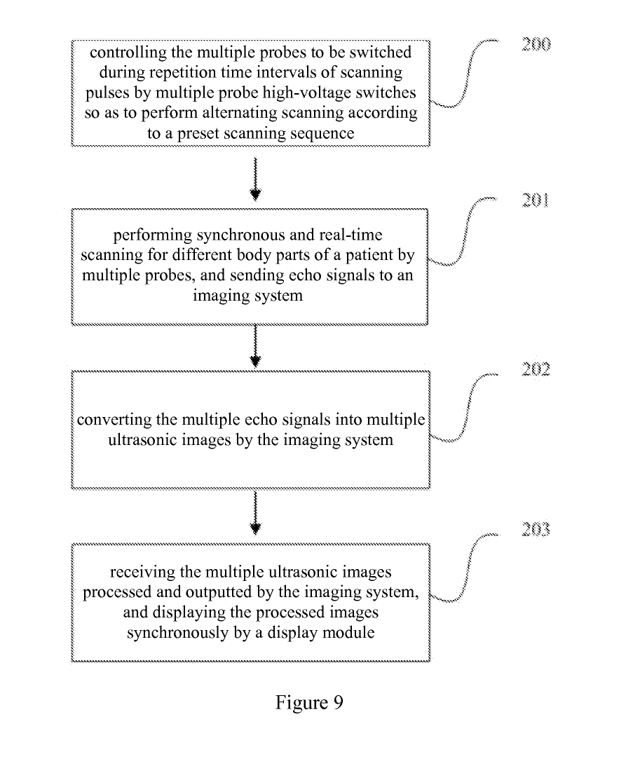

FIG. 9 is a flow chart for an ultrasonic diagnostic method according to a second embodiment. The second embodiment can include the following steps 200-203.

In step 200, multiple probes can be controlled to be switched during repetition time intervals of scanning pulses by multiple probe high-voltage switches, so that alternating scanning can be carried out for different body parts corresponding to different positions on a body surface of a patient according to a preset scanning sequence.

In step 201, the multiple probes can be used to perform synchronous and real-time scanning for the different body parts of the patient to obtain multiple echo signals, and the multiple echo signals can then be transmitted from the multiple probes to an imaging system.

In step 202, the multiple echo signals transmitted from the multiple probes can be converted into multiple ultrasonic images by the imaging system.

In step 203, when the multiple ultrasonic images are processed and outputted by the imaging system, a display module can receive the processed multiple ultrasonic images and display them synchronously.

The preset scanning sequence in the step 200 may be defined as follows: the alternating scanning may be successively performed per scan line by the multiple probes for the different body parts of the patient. Or, the preset scanning sequence can be defined as follows: the alternating scanning may be successively performed per frame by the multiple probes for the different body parts of the patient.

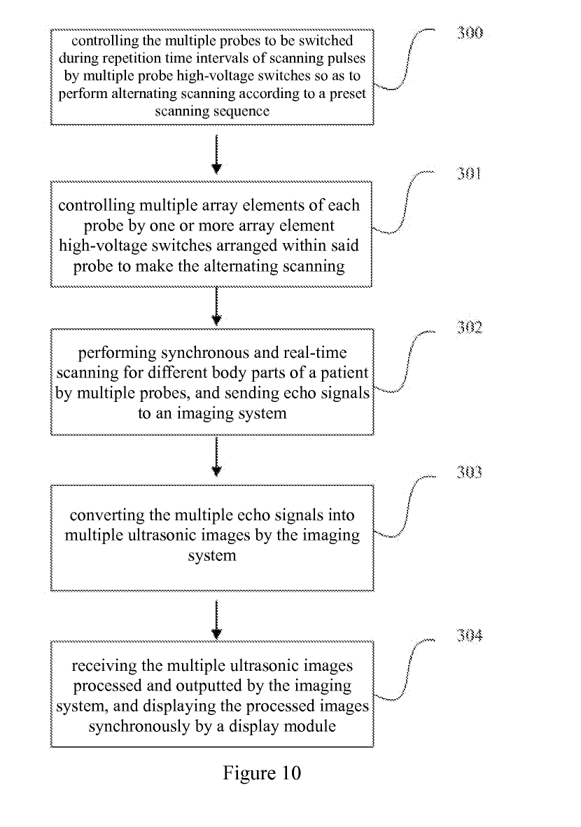

FIG. 10 is a flow chart for an ultrasonic diagnostic method according to a third embodiment of this disclosure.

The third embodiment can include the following steps 300-304.

In step 300, multiple probes can be controlled to be switched during repetition time intervals of scanning pulses by multiple probe high-voltage switches, so that alternating scanning can be carried out for different body parts corresponding to different positions on a body surface of a patient according to a preset scanning sequence.

In step 301, multiple array elements of each probe can be controlled by one or more array element high-voltage switches arranged within the probe to make the alternating scanning for the body part corresponding to the position on the body part where each probe is attached.

In some embodiments, the probe high-voltage switches and the array element high-voltage switches can be controlled by a control circuit.

In step 302, the multiple probes can be used to perform synchronous and real-time scanning for the different body parts corresponding to the different positions on the body surface of the patient to obtain multiple echo signals, and the multiple echo signals can then be transmitted from the multiple probes to an imaging system.

In step 303, the multiple echo signals transmitted from the multiple probes can be converted into multiple ultrasonic images by the imaging system.

In step 304, when the multiple ultrasonic images are processed and outputted by the imaging system, a display module can receive the processed multiple ultrasonic images and display them synchronously.

In the above-described three embodiments, the following method may be used for converting the multiple echo signals transmitted from the multiple probes into the multiple ultrasonic images by the imaging system:

performing digital processing on the multiple echo signals to obtain digital processing signals, and obtaining the multiple ultrasonic images according to the digital processing signals and a selected imaging mode. The imaging mode supported in the imaging system may be at least one of B imaging mode, M imaging mode, color imaging mode, pulse wave imaging mode, elasticity imaging mode, 3D imaging mode and 4D imaging mode.

Further details can be referred to the descriptions for FIGS. 2-7, which may not be repeated here.

In various embodiments of this disclosure, multiple slots connected with multiple probes can be arranged on the ultrasonic diagnostic device. The probes can realize the synchronous and real-time scanning so as to simultaneously perform ultrasonic scanning and monitoring on multiple body parts of a test subject.

The probes used in the embodiments of this disclosure can be attached to a patient's body surface for a long time. This can ensure that each scanning for the respective probe is made along the same section, thereby obtaining more accurate ultrasonic image and avoiding sound power risk caused by continuous scanning.

It should be understood for the ordinary skilled person in the art that all or partial processes in the above-described exemplary methods can be realized by instructions of computer programs on the relevant hardware. These programs can be stored within computer readable storage media. During their execution process, there may be some processes mentioned in the embodiments of those methods above. The storage medium can be magnetic disk, light disk, read only memory (ROM) or random access memory (RAM). The coupling referred in this disclosure can include contacting and non-contacting connection mode for signal/energy transmission. Although a monitoring host is defined in this disclosure, it should be understood that an ultrasonic host and a monitoring module integrated into the ultrasonic host can be used for achieving the same object. Also, an ultrasonic module and a monitoring module can be integrated into some other medical equipment or systems together. For example, the ultrasonic module and the monitoring module can be integrated into a CT device, an MRI device and so on.

The embodiments described above are preferred embodiments of this disclosure, which should not be used to limit the scope of the claims of this disclosure. Therefore, some equivalent changes made based on the claims of this disclosure should still fall within the scope of this disclosure.

1. An ultrasonic diagnostic device, comprising a display module, an imaging system and multiple probes, wherein:
the multiple probes are configured to be attached to different positions on a body surface of a patient; the multiple probes are configured to perform synchronous and real-time scanning for different body parts corresponding to the different positions on the body surface of the patient to obtain multiple echo signals, and the multiple echo signals are transmitted from the multiple probes to the imaging system;
the imaging system converts the multiple echo signals into multiple ultrasonic images; and
the display module, which is coupled to the imaging system, receives the multiple ultrasonic images processed and outputted by the imaging system, and displays the processed ultrasonic images synchronously.

2. The ultrasonic diagnostic device of claim 1, wherein each probe is tightly attached to a respective fixed position on the body surface of the patient, so that the scanning is performed for the patient at the respective fixed position along a same section.

3. The ultrasonic diagnostic device of claim 1, further comprising:
one or more slots for insertion connection with the multiple probes; and
one or more probe high-voltage switches for controlling the multiple probes to be switched during repetition time intervals of scanning pulses, such that alternating scanning is performed for the different body parts corresponding to the different positions on the body surface of the patient according to a preset scanning sequence;
wherein a quantity of the one or more probe high-voltage switches is equal to that of the one or more slots.

4. The ultrasonic diagnostic device of claim 3, wherein a quantity of the multiple probes is greater than or equal to that of the one or more slots.

5. The ultrasonic diagnostic device of claim 3, wherein the preset scanning sequence is defined as follows: the alternating scanning is successively performed per scan line by the multiple probes for the different body parts of the patient.

6. The ultrasonic diagnostic device of claim 5, wherein each probe comprises multiple array elements and one or more array element high-voltage switches corresponding to the multiple array elements; the array elements of each probe are controlled by the one or more corresponding array element high-voltage switches to make the alternating scanning for the body part corresponding to the position on the body surface where each probe is respectively attached.

7. The ultrasonic diagnostic device of claim 3, wherein the preset scanning sequence is defined as follows: the alternating scanning is successively performed per frame by the multiple probes for the different body parts of the patient.

8. The ultrasonic diagnostic device of claim 7, wherein each probe comprises multiple array elements and one or more array element high-voltage switches corresponding to the multiple array elements; the array elements of each probe are controlled by the one or more corresponding array element high-voltage switches to make the alternating scanning for the body part corresponding to the position on the body surface where each probe is respectively attached.

9. The ultrasonic diagnostic device of claim 8, wherein the probe high-voltage switch and the array element high-voltage switches are controlled by a control circuit.

10. The ultrasonic diagnostic device of claim 9, wherein the imaging system is used for performing digital processing on the multiple echo signals to obtain digital processing signals, and for obtaining the multiple ultrasonic images based on the digital processing signals and a selected imaging mode;
the imaging mode supported in the imaging system is at least one of B imaging mode, M imaging mode, color imaging mode, pulse wave imaging mode, elasticity imaging mode, three-dimensional imaging mode and four-dimensional imaging mode.

11. The ultrasonic diagnostic device of claim 10, further comprising an operation panel for receiving a triggering signal;
the display module comprises multiple display windows; the multiple display windows are used for displaying the multiple ultrasonic images in a real-time and synchronous way when the operation panel receives the triggering signal, wherein the multiple ultrasonic images are obtained by the imaging system according to the multiple echo signals and the selected imaging mode.

12. An ultrasonic diagnostic method, comprising:
performing synchronous and real-time scanning for different body parts corresponding to different positions on a body surface of a patient by multiple probes to obtain multiple echo signals, and transmitting the multiple echo signals from the multiple probes to an imaging system;
converting the multiple echo signals into multiple ultrasonic images by the imaging system; and
receiving the multiple ultrasonic images processed and outputted by the imaging system, and displaying the processed ultrasonic images synchronously by a display module.

13. The ultrasonic diagnostic method of claim 12, further comprising:
controlling the multiple probes to be switched during repetition time intervals of scanning pulses by multiple probe high-voltage switches, such that alternating scanning is carried out for the different body parts corresponding to the different positions on the body surface of the patient according to a preset scanning sequence.

14. The ultrasonic diagnostic method of claim 13, wherein the preset scanning sequence is defined as follows: the alternating scanning is successively performed per scan line by the multiple probes for the different body parts of the patient.

15. The ultrasonic diagnostic method of claim 13, wherein the preset scanning sequence is defined as follows: the alternating scanning is successively performed per frame by the multiple probes for the different body parts of the patient.

16. The ultrasonic diagnostic method of claim 13, further comprising:
controlling multiple array elements of each probe by one or more array element high-voltage switches arranged within said each probe to make the alternating scanning for the body part corresponding to the position on the body surface where each probe is attached.

17. The ultrasonic diagnostic method of claim 14, further comprising:
controlling multiple array elements of each probe by one or more array element high-voltage switches arranged within said each probe to make the alternating scanning for the body part corresponding to the position on the body surface where each probe is attached.

18. The ultrasonic diagnostic method of claim 17, wherein the probe high-voltage switches and the array element high-voltage switches are controlled to be switched by a control circuit.

19. The ultrasonic diagnostic method of claim 18, wherein converting the multiple echo signals into the multiple ultrasonic images by the imaging system comprises:
performing digital processing on the multiple echo signals to obtain digital processing signals, and obtaining the multiple ultrasonic images based on the digital processing signals and a selected imaging mode;
the imaging mode supported in the imaging system is at least one of B imaging mode, M imaging mode, color imaging mode, pulse wave imaging mode, elasticity imaging mode, three-dimensional imaging mode and four-dimensional imaging mode.
